(19) United States
(12) Patent Application Publication
(10) Pub. No.: US 2006/0136147 A1
(43) Pub. Date: Jun. 22, 2006

(54) Biological relationship event extraction system and method for processing biological information

(76) Inventors: Hyun-Chul Jang, Daejon-city (KR); Hyun-Sook Lee, Daejon-city (KR); Jae-Soo Lim, Daejon-city (KR); Soo-Jun Park, Seoul (KR); Seon Hee Park, Daejon-city (KR)

Correspondence Address: LADAS & PARRY LLP, 224 SOUTH MICHIGAN AVENUE, SUITE 1600, CHICAGO, IL, 60604, US

(21) Appl. No.: 11/304,030

(22) Filed: Dec. 15, 2005

(30) Foreign Application Priority Data
Dec. 20, 2004 (KR) 10-2004-0109046

(51) Int. Cl. G01N 33/48

(52) U.S. Cl. 702/20

(57) ABSTRACT

A biological relationship extraction system including a biological named entity substitution unit substituting a biological named entity in a biological document with a predetermined substitution name; a structure analyzing unit parsing the biological named entity in the biological document containing the substituted biological named entity; a relationship analyzing unit analyzing a relationship between biological named entities from the biological literature parsed by the structure analyzing unit and selecting relationship candidates; a relationship determining unit determining whether the relationship candidates delivered from the relationship analyzing unit are biologically meaningful and determining a relationship between biological named entities; and a biological named entity assignment storage unit storing the biological named entity and a substitution name corresponding to the biological named entity and providing a substitution name or a biological named entity.

### CROSS-REFERENCE TO RELATED APPLICATION

This application claims priority to and the benefit of Korean Patent Application 10-2004-0109046 filed in the Korean Intellectual Property Office on Dec. 20, 2004, the entire content of which, is incorporated herein by reference.

### BACKGROUND OF THE INVENTION

(a) Field of the Invention

The present invention relates to a biological relationship extraction system and a method for processing biological information. In particular, the biological relationship extraction system and the method for processing biological information searches a relationship between biological named entities extracted from biological information literature.

(b) Description of the Related Art

In recent years, vast amounts of biological literature that bears biological information have been published through the efforts of active studies in biology. Thus, a method for automatically extracting and processing useful information from the biological information-bearing literature is required.

In general, extraction of the biological information from the biological literature is purposed to recognize subjects of information within the literature and relationship between the subjects. It is also purposed to understand the biological process.

Thus, a method for recognizing a biological named entity as a subject and relationship information between the biological named entities in the biological information-bearing literature is required.

U.S. Pat. No. 6,539,376 (entitled “System and method for the automatic mining of new relationships”) disclosed a system for automatically extracting and classifying relationships by applying lexicographic and statistical techniques from a large text database of unstructured information. However, the system is not suitable for identifying relationship information between biological named entities.

A method for extraction information about specific functions between proteins only (e.g., interaction, activity, combination response, etc.) is typically used for recognizing a biological information relationship. This method is focused on a portion of functions between a specific protein and another protein within a limited protein domain. Thus, the information has a drawback of extracting limited information since the information is extracted according to a predefined rule.

Toshihide Ono disclosed a method for extracting information about proteins from biological literature and recognizing four types of relationships between proteins in “Automated Extraction of Information on Protein-protein Interactions from the Biological Literature (Bioinformatics, VOL. 17, NO. 22001, February. 2001).” However, the method does not sufficiently identify all kinds of relationships between biological entities.

According to another method disclosed by Gondy Leroy and Hsinchun Chen entitled “Filling Preposition-based Templates to Capture Information from Medical Abstracts (PSB, Proceedings 2002, 350-361, January 2002)”, three templates are built for extracting a sentence that may bear a relationship is extracted from biological literature, retrieving a main verb close to a preposition, and extracting a gene and a protein functioning as a subject or an object of the main verb in the sentence to identify relationships between biological named entities. However, this method does not cover all kinds of relationships between biological named entities.

As described, it is difficult to extract various relationships between biological named entities from the biological literature due to complicated notations of biological named entities.

Although a new technology employing a grammatical and statistical method has been developed, it is difficult to apply grammatical principles and build a corpus because of complicated characteristics of the biological literature.

The above information disclosed in this Background of the Invention section is only for enhancement of understanding of the background of the invention and therefore, it should not be understood that all the above information forms the prior art that is already known in this country to a person or ordinary skill in the art.

### SUMMARY OF THE INVENTION

It is an advantage of the present invention to provide a biological relationship extraction system for extracting biological named entities from a massive amount of biological literature and processing biological information.

It is another advantage of the present invention to provide a biological relationship extraction system for extracting biological named entities from a massive amount of biological literature and analyzing relationships between biological named entities.

It is another advantage of the present invention to provide a method for extracting biological named entities from a massive amount of biological literature and processing biological information.

In one aspect of the present invention, there is provided a biological relationship extraction system includes a biological named entity substitution unit, a structure analyzing unit, a relationship analyzing unit, a relationship determining unit, and a biological named entity assignment storage unit. The biological named entity substitution unit substitutes a biological named entity in a biological document with a predetermined substitution name. The structure analyzing unit parses the biological named entity in the biological document containing the substituted biological named entity. The relationship analyzing unit analyzes a relationship between biological named entities from the biological literature parsed by the structure analyzing unit and selects relationship candidates. The relationship determining unit determines whether the relationship candidates delivered from the relationship analyzing unit are biologically meaningful and determines a relationship between biological named entities. The biological named entity assignment storage unit stores the biological named entity and a substitution name corresponding to the biological named entity, and provides a substitution name or a biological named entity.

In another aspect of the present invention, there is provided a method for processing biological information. The method includes a) substituting a biological named entity with a predetermined substitution name; b) parsing biological literature in which the biological named entity is substituted; c) selecting relationship candidates between biological named entities using a biological named entity and a relative verb associated with the biological named entity; and d) selecting a biologically-meaningful relationship candidate from relationship candidates between biological named entities and determining a relationship between biological named entities.

### BRIEF DESCRIPTION OF THE DRAWINGS

FIG. 1 is a scheme diagram of a biological relationship extraction system according to a first exemplary embodiment of the present invention.

FIG. 2 illustrates a structure of a sentence tagged by a biological literature tagging unit of the biological relationship extraction system according to the first exemplary embodiment of the present invention.

FIG. 3 is a schematic diagram of a biological named entity substitution unit of the biological relationship extraction system according to the first exemplary embodiment of the present invention.

FIG. 4 illustrates a structure of a sentence substituted by the biological named entity substitution unit of the biological relationship extraction system according to the first exemplary embodiment of the present invention.

FIG. 5 is a schematic diagram of a structure analyzing unit of the biological relationship extraction system according to the first exemplary embodiment of the present invention.

FIG. 6 is a schematic diagram of a relationship searching unit of the biological relationship extraction system according to the first exemplary embodiment of the present invention.

FIG. 7 is a schematic diagram of a relationship determining unit of the biological relationship extraction system according to the first exemplary embodiment of the present invention.

FIG. 8 is a flowchart of a method for processing biological information according to a second exemplary embodiment of the present invention.

### DETAILED DESCRIPTION OF THE EMBODIMENTS

An embodiment of the present invention will hereinafter be described in detail with reference to the accompanying drawings.

In the following detailed description, only certain exemplary embodiments of the present invention have been shown and described, simply by way of illustration.

As those skilled in the art would realize, the described embodiments may be modified in various different ways, all without departing from the spirit or scope of the present invention.

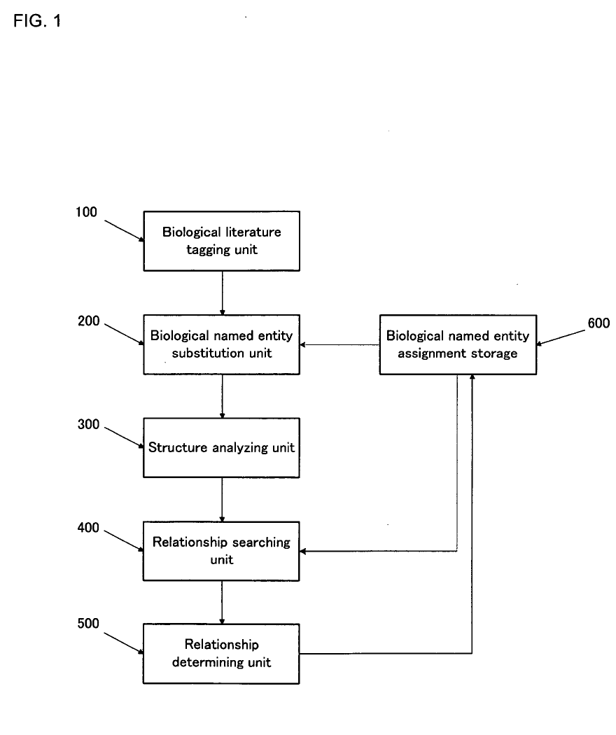

A biological relationship extraction system according to a first exemplary embodiment of the present invention will now be described with reference to FIG. 1.

FIG. 1 illustrates a biological relationship extraction system according to the first exemplary embodiment of the present invention.

The biological relationship extraction system includes a biological literature tagging unit 100, a biological named entity substitution unit 200, a structure analyzing unit 300, a relationship searching unit 400, a relationship determining unit 500, and a biological named entity assignment storage unit 600.

The biological literature tagging unit 100 extracts a sentence that bears biological information from biological literature, analyzes the sentence, and assigns tags to words in the sentence.

A method for assigning tags will be described using the following exemplary sentence: “Alzheimer's disease-associated amyloid beta interacts with the human serine protease HtrA2/Omi.”

First, each part-of-speech in the sentence is assigned a tag.

Alzheimer//NN 's//POS disease-associated//JJ amyloid//NN beta//NN interacts//VBZ with//IN the//DT human//NN serine//NN protease// HtrA2\/Omi//NN

Herein, NN denotes a noun, POS denotes a possessive, JJ denotes an adjective, VBZ denotes a verb, IN denotes a preposition, and DT denotes a definite article.

Next, a biological named entity is assigned a biological information-bearing tag (e. g., <NE> a biological named entity </NE>).

<NE> Alzheimer//NN 's//POS disease </NE> -associated//JJ <NE> amyloid//NN beta//NN </NE> interacts//VBZ with//IN the//DT human//NN serine//NN protease// <NE> HtrA2\/Omi//NN </NE>

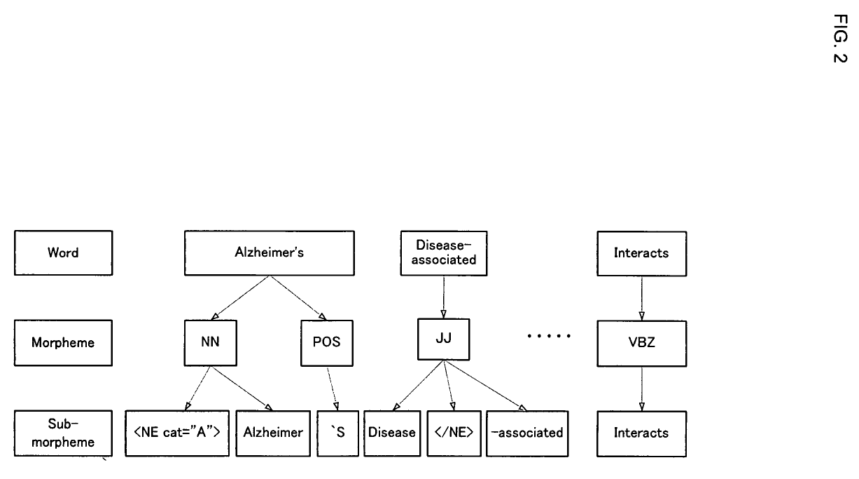

A method for tagging a sentence that bears biological information will now be described in more detail with reference to FIG. 2.

FIG. 2 illustrates a structure of a sentence tagged by the biological literature tagging unit of the biological relationship extraction system according to the first exemplary embodiment of the present invention.

As shown in FIG. 2, the part-of-speeches in the example sentence are first tagged with NN (noun), POS (possessive), JJ (adjective), and VBZ (verb), and then a biological named entity, “Alzheimer's disease”, is secondly assigned a biological information-bearing tag.

In this instance, each word in the sentence is assigned a tag according to a part-of-speech of the word, and the biological named entity, “Alzheimer's disease”, is additionally tagged with A.

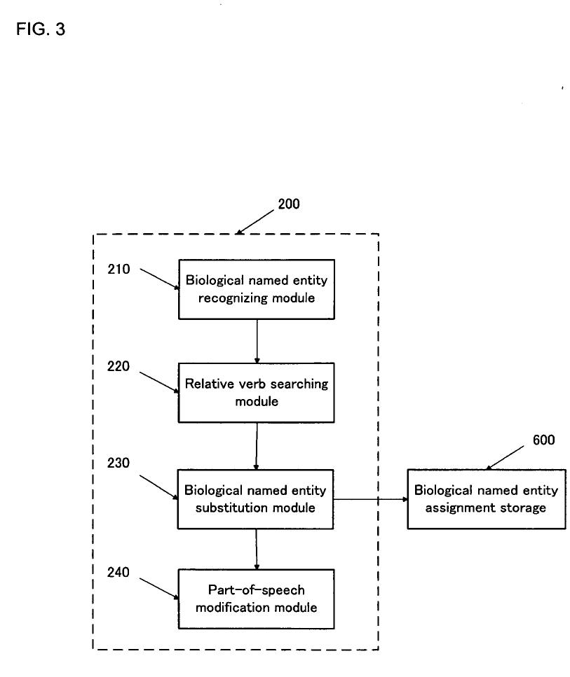

A configuration of the biological named entity substitution unit 200 of the biological relationship extraction system according to the first exemplary embodiment of the present invention will now be described with reference to FIG. 3.

FIG. 3 is a scheme diagram of the biological named entity substitution unit 200 of the biological relationship extraction system according to the first exemplary embodiment of the present invention.

The biological named entity substitution unit 200 receives tagged biological literature from the biological literature tagging unit 100, identifies a biological named entity from the biological information-bearing tag, and substitutes the biological named entity with a predetermined substitution name.

As shown in FIG. 3, the biological named entity substitution unit 200 includes a biological named entity recognizing module 210, a relative verb searching module 220, a biological named entity substitution module 230, and a part-of-speech modification module 240.

The biological named entity recognizing module 210 receives biological literature in which a biological named entity is tagged, searches the tagged biological named entity from the literature, and extracts the searched biological named entity.

The relative verb searching module 220 searches relative verbs associated with biological named entities in the biological literature, and checks which relative verb contains biologically-meaningful information in relationship with the extracted biological named entity among the searched relative verbs.

The biological named entity substitution module 230 divides the biological literature into sentences and substitutes biological named entities included in the separated sentences with predetermined substitution names. At this point, the biological named entity substitution module 230 checks whether an appropriate substitution name for the biological named entity exists in the biological named entity assignment storage unit 600. If one exists, the biological named entity substitution module 230 receives the appropriate substitution name and substitutes the biological named entity with the received substitution name.

If one does not exist, the biological named entity substitution module 230 generates a substitution name for the biological named entity.

In this instance, the biological named entity and the generated substitution name are stored in the biological named entity assignment storage unit 600.

The part-of-speech modification module 240 checks whether the sentence that includes the predetermined substitution name for the biological named entity is appropriate, and modifies part-of-speech tagging information.

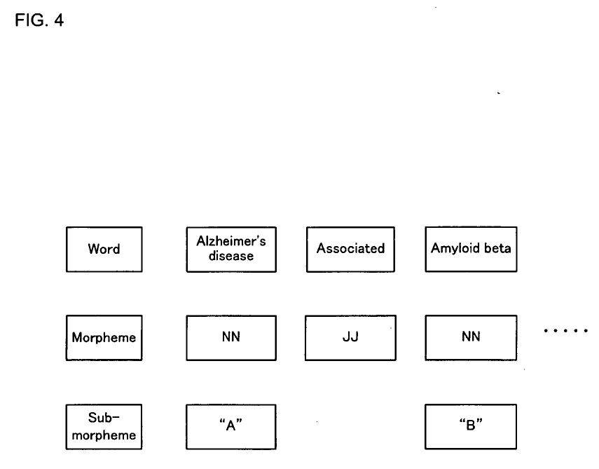

FIG. 4 illustrates a structure of a sentence substituted by the biological named entity substitution unit of the biological relationship extraction system according to the first exemplary embodiment of the present invention.

The above example sentence, “Alzheimer's disease-associated amyloid beta interacts with the human serine protease HtrA2/Omi”, is used again in FIG. 4.

As shown in FIG. 4, a biological named entity, “Alzheimer's disease” is a noun (NN), and is substituted with a substitution name A. Another biological named entity, “amyloid beta” is a noun, and is substituted with a substitution name B.

Although it is not shown in FIG. 4, biological named entities “human serine protease” and “HtrA2/Omi” may be respectively substituted with substitution names C and D, and thus the example sentence may be substituted into “NEA-associated NEB interacts with the NEC NED” by the biological named entity substitution module 230. No biological named entity is included in the substituted sentence.

In this instance, NE denotes a biological named entity.

In addition, the substituted sentence is modified into “JJ NN VBZ IN DT NN NN” by the part-of-speech modification module 240.

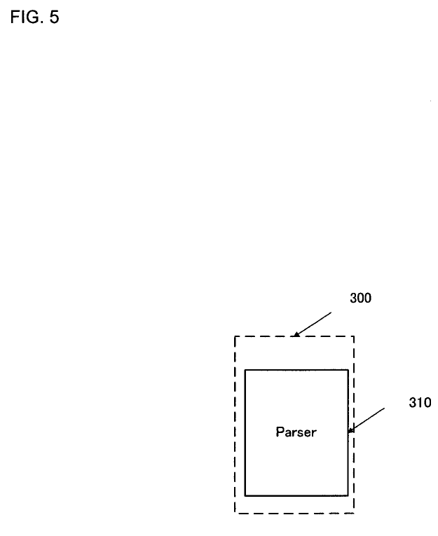

A configuration of the structure analyzing unit 300 of the biological relationship extraction system according to the first exemplary embodiment of the present invention will now be described with reference to FIG. 5.

FIG. 5 is a scheme diagram of the structure analyzing unit 300 of the biological relationship extraction system according to the first exemplary embodiment of the present invention.

As shown in FIG. 5, the structure analyzing unit 300 includes a parser 310.

The structure analyzing unit 300 uses the parser 310 to parse the substituted sentence delivered from the biological named entity substitution unit 200, analyzes a structure of the sentence, and expresses the sentence in a tree structure. The parser 310 could be a typical parser.

Performance of the parser 310 may be optimized because a complex sentence becomes a simple sentence by substituting a complex biological named entity with a simple substitution name using the biological named entity substitution unit 200 according to the first exemplary embodiment of the present invention.

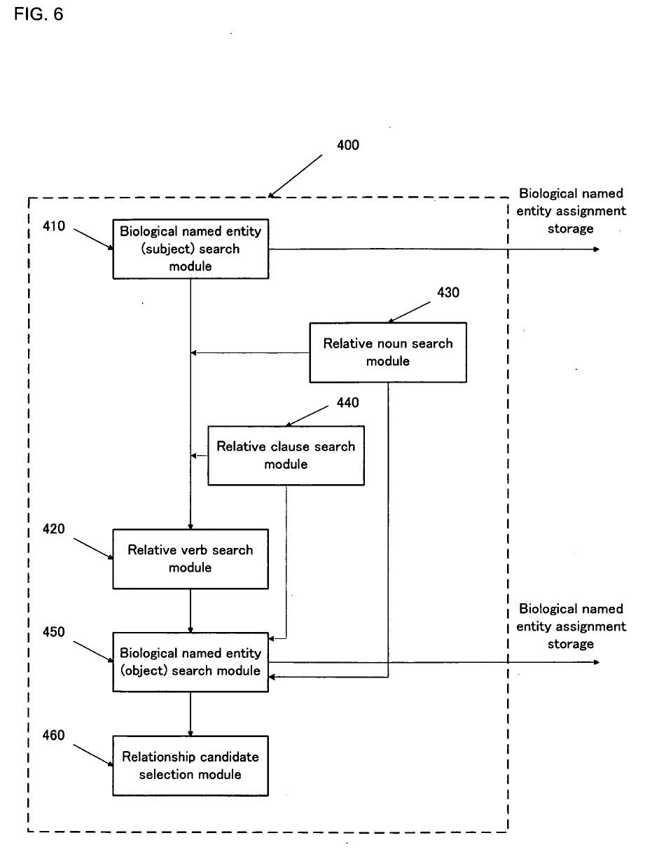

A configuration of a relationship searching unit 400 of the biological relationship extraction system according to the first exemplary embodiment of the present invention will now be described with reference to FIG. 6.

The relationship searching unit 400 analyzes the sentence parsed by the structure analyzing unit 300 and analyzes relationships between biological named entities using substitution names and biological named entities stored in the biological named entity assignment storage unit 600 such that the relationship searching unit 400 retrieves a relationship candidate. In more detail, the relationship searching unit 400 analyzes the parsed sentence, searches a biological named entity, searches a relative verb that is associated with the identified biological named entity, and searches another biological named entity that is associated with the identified relative verb. When the biological named entity, the relative verb, and another biological named entity that is associated with the relative verb are searched, the two biological named entities and the relative verb compose relationship information.

FIG. 6 is a scheme diagram illustrating an exemplary realization of the relationship searching unit 400 of the biological relationship extraction system according to the first exemplary embodiment of the present invention.

As shown in FIG. 6, the relationship searching unit 400 includes a biological named entity (subject) search module 410, a relative verb search module 420, a relative noun search module 430, a relative clause search module 440, a biological named entity (object) search module 450, and a relationship candidate selection module 460.

The biological named entity (subject) search module 410 receives the parsed sentence from the structure analyzing unit 300, recognizes a substitution name functioning as a subject in the parsed sentence, and extracts a biological named entity that corresponds to the substitution name from the biological named entity assignment storage unit 600. A substitution name functioning as a subject in a sentence generally includes a substitution name functioning as a subject in a relative clause included in the sentence.

The relative verb search module 420 searches a relative verb associated with the biological named entity extracted by the biological named entity (subject) search module 410. Herein, the relative verb includes all types of verbs such as a passive verb, a progressive verb, a past tense verb, a present tense verb, and so on, and a word directly and indirectly associated to the biological named entity.

The biological named entity (object) search module 450 searches a substitution name that functions as an object of the relative verb in the parsed sentence, and extracts a biological named entity that corresponds to the substitution name from the biological named entity assignment storage unit 600. A substitution name that functions as an object generally includes a substitution name that functions as an object in a sentence.

When the extracted biological named entity is associated with a noun form of the searched relative verb, the relative noun search module 430 searches whether another biological named entity is associated with the noun form. Herein, a noun form of a relative verb includes a participial form of the relative verb. In more detail, when the relative verb is “interact,” the noun form of the relative verb includes “interacting” and “interaction.”

When more than two biological named entities are associated with a noun form of a relative verb, the two biological named entities become candidates such that relationship information may be retrieved therefrom.

When a relative clause is associated to the extracted biological named entity rather than a relative verb is directly associated to the extracted biological named entity, the relative clause search module 440 searches a relative verb and a biological named entity in the relative clause. A relative clause could be identified by existence of a relative pronoun.

When more than two biological named entities are associated with one relative verb, the relationship candidate selection module 460 perceives that the two biological named entities are related to each other and selects them as relationship candidates. In particular, when the biological named entity extracted by the biological named entity (subject) search module 410, the relative verb associated with the extracted biological named entity and searched by the relative verb search module 420, and the biological named entity functioning as an object of the searched the relative verb exist, the subjective and objective biological named entities are selected as the relationship candidates.

Apart from the exemplary realization shown in FIG. 6, when a relative verb associated with a substitution name functioning as a subject in a biological information-bearing sentence is searched and a substitution name functioning as an object of the searched relative verb is searched, biological named entities that respectively correspond to the substitution name (subject) and the substitution name (object) may be selected as the relationship candidates according to another exemplary realization.

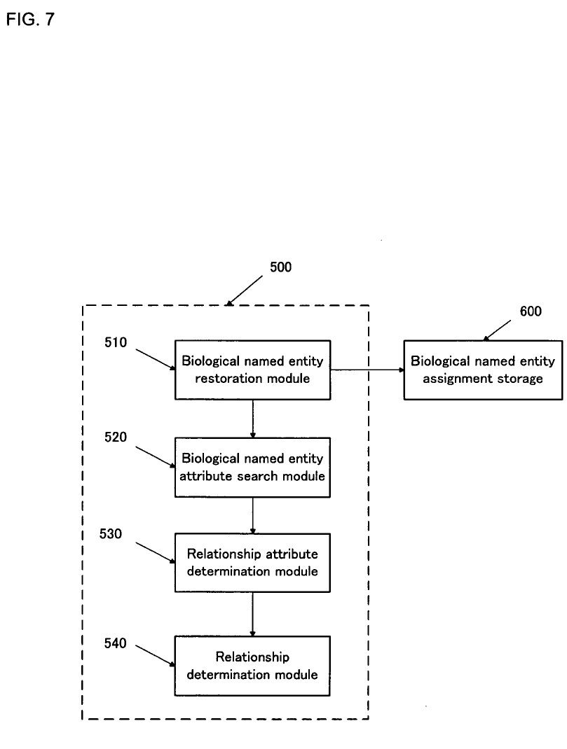

The relationship determining unit 500 of the biological relationship extraction system according to the first exemplary embodiment of the present invention will be described with reference to FIG. 7.

FIG. 7 is a scheme diagram of the relationship determining unit 500 of the biological relationship extraction system according to the first exemplary embodiment of the present invention.

The relationship determining unit 500 receives the relationship candidates selected by the relationship searching unit 400 and selects biologically-meaningful relationship candidates so as to determine a relationship between the biological named entities.

As shown in FIG. 7, the relationship determining unit 500 includes a biological named entity restoration module 510, a biological named entity attribute searching module 520, a relationship attribute determination module 530, and a relationship determination module 540.

The biological named entity restoration module 510 extracts a biological named entity that corresponds to a substitution name from the biological named entity assignment storage unit 600 and restores the biological named entity.

The biological named entity attribute search module 520 checks attributes of the restored biological named entity and assigns the attributes to the biological named entity. The attributes of the biological named entity may vary depending on the type of a biological object identified by the biological named entity. Herein, the type of the biological object includes a microscopic organism, deoxyribonucleic (DNA), ribonucleic acid (RNA), a protein, an amino acid, an enzyme, a coenzyme, a vitamin, and glucose, etc. An attribute of a biological named entity may be identified by a notation form of the biological named entity. In more detail, if a biological named entity ends with “-ase”, an attribute of the biological named entity is an enzyme.

The biological named entity attribute search module 520 includes a biological information database, and searches attributes of biological named entities by using the biological information database.

The relationship attribute determination module 530 compares an object of a biological named entity and a relative verb associated with the biological named entity with reference to attributes of the biological named entity assigned by the biological named entity attribute search module 520, and determines whether relationship candidates between biological named entities contain biologically-meaningful information.

For example, when relationship candidates are objects of biological named entities, and the biological named entities are respectively a DNA polymerase and a given DNA and a relative verb is “transcript”, the DNA polymerase and the given DNA provide biologically-meaningful information but the relative verb “transcript” is associated with RNA. Thus, the relationship candidates do not contain biologically-meaningful information. In this instance, when the relative verb is “polymerize”, this implies that the DNA polymerase polymerizes the given DNA, and accordingly the relationship candidates are determined to be biologically meaningful.

The relationship determination module 540 includes a database that stores biological knowledge determination rules, and determines whether attributes between biological named entities are biologically meaningful with reference to the biological knowledge determination rules. For example, the biological knowledge determination rules may include the above-mentioned examples, <DNA, polymerase> and <RNA, transcriptase>.

The relationship determination module 550 determines the relationship candidates, which are determined to be biologically meaningful by the relationship determination module 540, as a relationship of the biological named entities.

The biological named entity assignment storage unit 600 stores a biological named entity and its corresponding substitution name, and assigns an appropriate substitution name to a biological named entity or a biological named entity to a substitution name according to requests from the biological named entity substitution unit 200, the relationship searching unit 400, and the relationship determining unit 500. When an appropriate substitution name for a biological named entity does not exist in the biological named entity assignment storage unit 600, the biological named entity assignment storage unit 600 generates a substitution name and assigns it to the biological named entity. For this reason, the biological named entity assignment storage unit 600 may include a substitution name generation module.

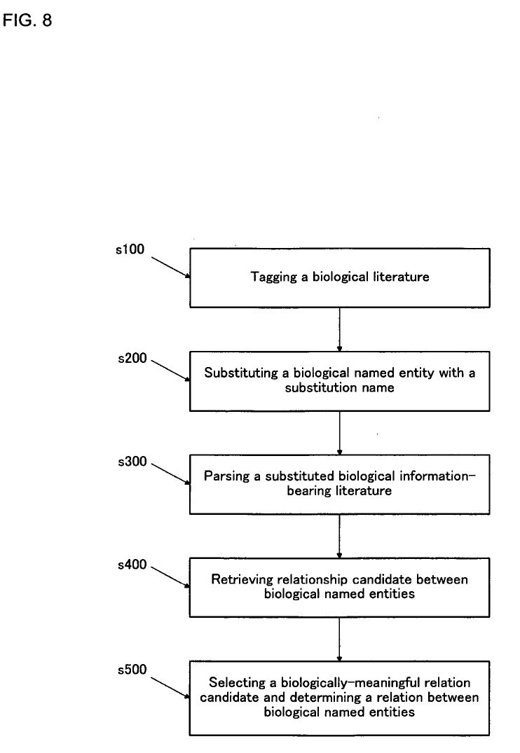

A method for searching biological information according to a second exemplary embodiment of the present invention will now be described with reference to FIG. 8.

A biological literature containing biological information is tagged in step s100. Tagging of the biological literature may include analyzing biological information-bearing sentences, assigning tags to words in the sentences, and assigning biological information-bearing tags to biological named entities.

The tagged biological literature is received and a biological named entity in the literature is substituted with a predetermined substitution name, in step s200.

In more detail, the biological named entity is searched in the tagged biological literature to substitute the biological named entity with the predetermined substitution name when the biological literature is received. A relative verb associated with the searched biological named entity is searched, and a biological named entity associated with the searched relative verb is substituted with the predetermined substitution name. Then part-of-speech tagging information is modified and biological named entities are substituted with predetermined substitution names in the substituted biological literature. Appropriateness of substituted sentences is checked and the part-of-speech tagging information is modified accordingly.

As an example of modifying the part-of-speech tagging information in the tagged biological literature, a biological named entity composed of several part-of-speech tags (e.g., <NE> Alzheimer//NN 's//POS disease </NE>) may be modified to one noun tag (NN) as shown in FIG. 4.

Words (e.g., -associated//JJ) associated with the biological named entity are separated and tagged with an appropriate part-of-speech tag (e.g., JJ). When an original biological named entity composed of at least one word is substituted with one substitution name, a part-of-speech tag assigned to an unnecessary word (e.g., a possessive case tag ‘POS’) is eliminated.

The biological literature in which biological named entities are substituted with predetermined substitution names is received and parsed in step s300.

The parsed biological document is received and a relationship between biological named entities is analyzed by using the biological named entities and a relative verb associated with the biological named entities such that relationship candidates between the biological named entities are selected in step s400.

In more detail, a biological named entity corresponding to a substitution name, which functions as a subject in the biological literature, is extracted and a relative verb associated with the biological named entity is searched.

A biological named entity corresponding to a substitution name, which functions as an object of the relative verb, is extracted, and relationship candidates of the two biological named entities (subject and object) are selected.

A biological named entity that corresponds to a substitution name, which functions as a subject in a parsed sentence, may be extracted according to another method for selecting relationship candidates. A relative verb associated with the biological named entity is searched.

A biological named entity corresponding to a substitution name that functions as an object of the searched relative verb is extracted, and then the biological named entities respectively function as the subject and the object are selected as the relationship candidates.

At this point, a noun associated with a biological named entity is checked to determine whether it is a noun form of a relative verb. If so, another biological named entity that is associated with the noun is searched.

When a relative clause is associated with the biological named entity, a biological named entity associated with a relative verb included in the relative clause is searched and the biological named entity associated with the relative clause and the biological named entity associated with the relative verb included in the relative clause are selected as relationship candidates.

The relationship candidates of the extracted biological named entities are received, and a relationship of biological named entities is determined by selecting biologically-meaningful relationship candidates in step s500.

In more detail, the biological named entity corresponding to the substitution name is extracted and restored, and biological attributes of the biological named entity are checked so as to determine whether the subjective biological named entity, the objective biological named entity, and the relative verb have a biologically-meaningful relationship with each other.

If they have the biologically-meaningful relationship, the relationship candidates are determined as a biological named entity relation. Otherwise, the relationship candidates are discarded.

According to the embodiments of the present invention, a relationship between biological named entities is automatically extracted and analyzed from a large amount of biological literature.

In addition, a biological named entity is substituted with a simple substitution name such that a complex sentence that bears biological information becomes a simple sentence. Accordingly, performance of a parser is optimized when it is used for analyzing a structure of the sentence. As a result, a vast amount of biological literature can be efficiently processed.

Further, reliability of a biological information processing result is enhanced by determining a biological meaning of a biological named entity relationship.

While this invention has been described in connection with what is presently considered to be practical exemplary embodiments, it is to be understood that the invention is not limited to the disclosed embodiments, but, on the contrary, is intended to cover various modifications and equivalent arrangements included within the spirit and scope of the appended claims.

What is claimed is:

1. A biological relationship extraction system comprising:
a biological named entity substitution unit substituting a biological named entity in a biological document with a predetermined substitution name;
a structure analyzing unit parsing the biological named entity in the biological document containing the substituted biological named entity;
a relationship analyzing unit analyzing a relationship between biological named entities from the biological literature parsed by the structure analyzing unit and selecting relationship candidates;
a relationship determining unit determining whether the relationship candidates delivered from the relationship analyzing unit are biologically meaningful and determining a relationship between biological named entities; and
a biological named entity assignment storage unit storing the biological named entity and a substitution name corresponding to the biological named entity, and providing a substitution name or a biological named entity.

2. The biological relationship extraction system of claim 1, further comprising:
a biological literature tagging unit analyzing a biological information-bearing sentence, assigning a tag to each word in the sentence, and assigning a biological information-bearing tag to a word corresponding to a biological named entity,
wherein biological literature having been assigned tags by the biological literature tagging unit is input to the biological named entity substitution unit.

3. The biological relationship extraction system of claim 1, wherein the biological named entity substitution unit comprises:
a biological named entity recognizing module recognizing a biological named entity from the biological literature; and
a biological named entity substitution module receiving a request for a substitution name that corresponds to a biological named entity, and substituting the biological named entity with a substitution name received from the biological named entity assignment storage unit.

4. The biological relationship extraction system of claim 3, wherein the biological named entity substitution unit further comprises a part-of-speech tagging modification module modifying part-of-speech tagging information of a substituted sentence.

5. The biological relationship extraction system of claim 1, wherein the relationship analyzing unit comprises:
a relative verb searching module receiving a parsed sentence from the structure analyzing unit, and searching a relative verb associated with a substitution name that corresponds to a biological named entity; and
a relationship candidate selection module selecting more than two biological named entities as relationship candidates when the more than two biological named entities are associated with one relative verb.

6. The biological relationship extraction system of claim 1, wherein the relationship analyzing unit comprises:
a first biological named entity recognizing module requesting a biological named entity corresponding to a substitution name from the biological named entity assignment storage unit, the substitution name functioning as a subject in a parsed sentence;
a relative verb searching module searching a relative verb associated with a substitution name which functions as a subject in a parsed sentence;
a second biological named entity recognizing module requesting a biological named entity corresponding to a substitution name from the biological named entity assignment storage, the substitution name functioning as an object of the relative verb searched by the relative verb searching module; and
a relationship candidate selection module selecting the biological named entity searched by the first biological named entity recognizing module, the biological named entity recognized by the second biological named entity recognizing module, and the relative verb searched by the relative verb searching module as relationship candidates.

7. The biological relationship extraction system of claim 5, further comprising, a relative noun searching module searching another biological named entity associated with the noun form of the relative verb when a relative verb associated with the biological named entity is a noun form of the relative verb.

8. The biological relationship extraction system of claim 5, further comprising a relative clause searching module searching a biological named entity and a relative verb that compose the relative clause when a relative clause is associated with the biological named entity.

9. The biological relationship extraction system of claim 1, wherein the relationship determining unit comprises:
a biological named entity attribute search module checking attributes of a biological named entity included in the relationship candidates and assigning the attributes to the biological named entity; and
a relationship attribute determination module comparing attributes assigned by the biological named entity attributes module, and determining whether the relationship candidates are biologically meaningful.

10. The biological relationship extraction system of claim 9, wherein the biological named entity attribute search module comprises a biological information database storing attributes of biological named entities.

11. The biological relationship extraction system of claim 9, wherein the relationship attribute determination module comprises a biological knowledge determining rule and a biological knowledge determining database providing a biological knowledge rule for the biological named entity.

12. The biological relationship extraction system of claim 1, wherein the biological named entity assignment storage unit comprises a substitution name generation module generating a substitution name corresponding to a biological named entity which is not stored in the biological named entity assignment storage.

13. A method for processing biological information, comprising:
a) substituting a biological named entity with a predetermined substitution name;
b) parsing biological literature in which the biological named entity is substituted;
c) selecting relationship candidates between biological named entities using a biological named entity and a relative verb associated with the biological named entity; and
d) selecting a biologically-meaningful relationship candidate from relationship candidates between biological named entities and determining a relationship between biological named entities.

14. The method of claim 13, further comprising:
analyzing a sentence bearing biological information and assigning a tag to each word in the sentence; and
assigning a biological information-bearing tag to a word corresponding to the biological named entity.

15. The method of claim 13, wherein c) comprises:
analyzing a parsed sentence, and searching a substitution which functions as a subject in the parsed sentence;
searching a relative verb associated with the substitution name functioning as the subject;
searching a substitution name functioning as an object of the searched relative verb; and
searching biological named entities respectively corresponding to the substitution name functioning as the subject and the substitution name functioning as the object as relationship candidates when the substitution name functioning as the object of the relative verb exists.

16. The method of claim 13, wherein c) comprises:
checking whether a noun associated with the biological named entity is a noun form of a relative verb; and
recognizing another biological named entity associated with the noun when the noun is the noun form of the relative verb.

17. The method of claim 13, wherein c) comprising:
searching a relative clause associated with the biological named entity; and
searching a biological named entity associated with a relative verb within the relative clause and selecting a biological named entity associated with the relative verb in the relative clause and the searched biological named entity as relationship candidates when a relative clause is associated with the biological named entity.
